(19) United States
(12) Patent Application Publication
(10) Pub. No.: US 2005/0089489 A1
(43) Pub. Date: Apr. 28, 2005

(54) Composition for exfoliation agent effective in removing resist residues

(76) Inventor: Melvin K. Carter, Los Gatos, CA (US)

Correspondence Address: MORGAN LEWIS & BOCKIUS LLP, 1111 PENNSYLVANIA AVENUE NW, WASHINGTON, DC, 20004, US

(21) Appl. No.: 10/689,616

(22) Filed: Oct. 22, 2003

(51) Int. Cl. F23J 1/00; A61K 7/06

(52) U.S. Cl. 424/70.1; 134/26; 134/2

(57) ABSTRACT

Improved fluoride-based compositions effective in exfoliating resist residues resulting from dry etching and plasma ashing are disclosed. An excellent anti-corrosion effect, as well as resist residue exfoliation effect, can be achieved using the disclosed compositions.

### FIELD OF THE INVENTION

The present invention relates to a composition of exfoliation agent to be used to remove resist residues. More specifically, the invention is appropriately used in the production of semiconductor elements such as IC and LSI, and those used in the liquid crystal display devices. The composition can exfoliate resist residues at a low temperature and in a short period of time, while being highly safe and easy to use.

### BACKGROUND OF THE INVENTION

Cleaning solvents are used throughout industry. These solvents are made from various organic and inorganic materials forming compositions differing in functionality and effectiveness. In order for cleaning solvents to be effective, the cleaning solvent, the material to be removed, and the surrounding material or substrate must be examined to insure that the material or residue to be removed can be dissolved, solvated, or removed by the cleaning solvent without damaging the surrounding materials. Several factors, such as pH, polarity, chemical reactivity, and chemical compatibility must be considered when selecting a cleaning solvent. Other factors must also be considered when utilizing cleaning solvents, such as environmental regulations, safety concerns, and cost.

Fluoride containing chemistries have been used for many years with prime silicon wafers (wafers that have not yet undergone ion implantation or device construction) in the semiconductor industry. Normally the fluoride chemistry (usually dilute hydrofluoric acid) is used as the last process step in the sequence called “RCA rinses.” The substrate is often contaminated from previous process steps with monolayer amounts of metal, anions and/or organic contaminants or surface residues (particles). These contaminants have been shown to have a significant impact on the electrical integrity of simple test device structures and these structures need to be cleaned efficiently without impairing their integrity. Such cleaning methods could include techniques discussed in the technical literature, for example, mt. Conf. On Solid State Devices and Materials, 1991, pp. 484-486, Kujime, T. et al., Proc. of the 1996 Semi. Pure Water and Chemicals, pp. 245-256, and Singer, P., Semi. International, p. 88, October 1995.

Patents that teach methods for cleaning prime wafers with low pH solutions include U.S. Pat. Nos. 5,560,857; 5,645,737; 5,181,985; 5,603,849; and 5,705,089.

Using fluoride chemistries (usually HF) as a final RCA cleaning step will cause the silicon wafer surface to be in a hydrophobic state (the surface is covered with Si—H groups) which will repel water. During this step a certain proportion of the wafer surface is dissolved (removed). Unless the conditions are carefully monitored (time, temperature, solution composition) the substrates can be damaged, as reported by Rafols, C. et al., J. Electroanalytic Chem., 433, pp. 77-83, 1997. Numerous compositions combine water and organic solvents. The water concentration in these solutions is very critical. Silica oxide has an etch rate of 21 Å/min (@ 25° C.) in HF/water, but in isobutanol the rate was reduced to 2.14 Å/min and even lower in acetone (an aprotic solvent) the rate was only 0.12 Å/min, as reported at NSF/SRC Eng. Res. Center, Environmentally Benign Semiconductor Manufacturing, Aug. 5-7, 1998, Stanford University.

After the Front End of Line (FEOL) cleaning process the wafer proceeds to the typical Back End of Line (BEOL) manufacturing process for a semiconductor devices, in which the devices might be dynamic random access memories (DRAMs), static random access memories (SRAMs), logic, electrically programmable read only memories (EPROMs), complementary metal on silicon (CMOS), and the like. Etching fabrication technology using chemical reactions (liquid or plasma) has been used as a method of forming a wiring structure on such semiconductor substrates.

A photoresist film is deposited on the wafer to form a mask, then a substrate design is imaged on the film layer, baked, and the undeveloped image is removed with a developer. The remaining image is then transferred to the underlying material through etching (either a dielectric or metal) with reactive etching gases promoted with plasma energy.

The etchant gases selectively attack the unprotected area of the substrate. Liquid or wet etching chemistries have been used extensively over the years to etch metals, oxides and dielectrics. These chemistries can be very aggressive and can result in isotropic etching (etching equally in all directions).

Increasingly, plasma etching, reactive ion etching or ion milling are used, and such etching processes produce undesirable by-products from the interaction of the plasma gases, reacted species and the photoresist. The composition of such by-products is generally made up of the etched substrates, underlying substrate, photoresist and etching gases. The formation of such by-products is influenced by the type of etching equipment, process conditions and substrates utilized. These by-products are generally referred to as “sidewall polymer,” “veil,” or “fences” and cannot be removed completely by either oxygen plasma or conventional solvents. Examples of alkaline/solvent mixture types of photoresist strippers which are known for use in stripping applications include dimethylacetamide or dimethylformamide and alkanolamines as described in U.S. Pat. Nos. 4,770,713 and 4,403,029; 2-pyrrolidone, dialkylsulfone and alkanolamines as described in U.S. Pat. Nos. 4,428,871, 4,401,747, and 4,395,479; and 2-pyrrolidone and tetramethylammonium hydroxide as described in U.S. Pat. No. 4,744,834. Such stripping compositions, however, have only proven successful in cleaning “sidewall polymer” from the contact openings and metal line etching in simple microcircuit manufacturing involving a single layer of metal process when the metal structure involves mainly Al—Si or Al—Si—Cu and the “sidewall polymer” residue contains only an organometallic compound with aluminum.

If etching residue is not removed from the substrate, the residue can interfere with subsequent processes involving the substrate. The need to effectively remove etching residue and photoresist from a substrate becomes more critical as the industry progresses into submicron processing techniques. The requirement for cleaning solutions that remove all types of residue generated as a result of plasma etching of various types of metals, such as aluminum, aluminum/silicon/copper, titanium, titanium nitride, titanium/tungsten, tungsten, silicon oxide, polysilicon crystal, etc., while not corroding the underlying metal presents a need for more effective chemistry in the processing area. The effect of poor cleaning results in low device yield, low device reliability, and low device performance.

Also, if the components in these residues are not removed or neutralized in some manner then the residues will absorb moisture and form acidic species that can corrode the metal structures. The resultant acid corrodes wiring materials to bring about an adverse effect such as an increase in electrical resistance and wire disconnection. Such problems frequently occur, in particular in aluminum and aluminum alloys generally used as wiring material. The wafer substrate in contact with acidic materials, if not controlled, can destroy the metal structures. Following completion of the etching operation it is necessary that the post-etch resist mask be removed from the protective surface to permit finishing operations.

It is desirable to develop an improved cleaning composition to remove the organic polymeric substance from a coated inorganic substrate without corroding, dissolving or dulling the metal circuitry or chemically altering the wafer substrate.

Sidewall residues have been removed with either acidic organic solvents or alkaline organic solvents. The acidic solvents are generally composed of phenolic compounds or chloro-solvent and/or an aromatic hydrocarbon and/or alkylbenzenesulfonic acids. These formulations generally need to be used at temperatures up to and beyond 100° C. These chemistries normally need to be rinsed with isopropanol.

In addition, stripping compositions used for removing photoresist coatings and cleaning composition for removing post-etch residue have for the most part been highly flammable, generally hazardous to both humans and the environment, and comprise reactive solvent mixtures exhibiting an undesirable degree of toxicity. Moreover, these compositions are not only toxic, but their disposal is costly since they might have to be disposed of as a hazardous waste. In addition, these compositions generally have severely limited bath life and, for the most part, are not recyclable or reusable.

The photoresist around the contact hole of common interlayer dielectrics, TEOS (tetraethylorthosilicate) and boron phosphosilicate glass (BPSG), which are commonly used in ultra large scale integration (ULSI) structures for better conformity of step coverage, is usually removed with HF solutions. It is not uncommon for the HF to also attack the dielectric material. Such attack is not desirable (see Lee, C. and Lee, 5, Solid State Electronics, 4, pp. 92 1-923 (1997)).

Dilute hydrofluoric acid solutions can under certain conditions remove the sidewall polymers by aggressively attacking the via sidewall of the dielectric and therefore changing the dimensions of the device, as taught by Ireland, P., Thin Solid Films, 304, pp. 1-12 (1997), and possibly the dielectric constant. Previous chemistries that contain HF, nitric acid, water and hydroxylamine are aggressive enough to etch silicon, as taught by U.S. Pat. No. 3,592,773 issued to A. Muller. Recent information also indicates that the dilute HF solutions can be ineffective for cleaning the newer CFx etch residues, as taught by K. Ueno et al., “Cleaning of CHF₃ Plasma-Etched SiO₂/SiN/Cu Via Structures with Dilute Hydrofluoric Acid Solutions,” J. Electrochem. Soc., vol. 144(7), 1997. Contact holes opened on to the TiSi₂ have also been difficult to clean with HF solutions since there appears to be an attack of the underlying TiSi₂ layer. There may also be difficulty with mass transport of the chemicals in the narrow hydrophilic contact holes, as taught by Baklanov, M. R. et al., Proc. Electrochem. Soc., 1998, 97-35, pp. 602-609.

Recently, fluoride-based chemistries have been used in limited cases to remove post etch residues. Many of these compositions contain fluoride components, specifically hydrogen fluoride. In addition these compositions might contain strong caustic chemicals (choline-derivatives, tetraalkyl ammonium hydroxide, ammonium hydroxide) such as disclosed in U.S. Pat. No. 5,129,955; U.S. Pat. No. 5,563,119; or U.S. Pat. No. 5,571,447, or might use a two-phase solvent system, which contains one phase with hydrofluoric acid and water while a second phase contains an organic solvent such as ketones or ethers, such as disclosed in U.S. Pat. No. 5,603,849. Other formulations include hydroxylamine and ammonium fluoride (U.S. Pat. No. 5,709,756, issued to Ward). Additional examples include quaternary ammonium salt and fluoride based compositions, as disclosed in published European Application No. 0662705, and organocarboxylic ammonium salt or amine carboxylate and fluoride based compositions, as disclosed in U.S. Pat. No. 5,630,904.

Other methods for cleaning metal and metal oxide residues on wafers include spraying water vapor into the plasma ashing chamber followed by introducing fluorine containing gases (hydrofluoric acid), as disclosed in U.S. Pat. No. 5,181,985, or a liquid containing hydrofluoric acid. ammonium fluoride and water with a pH between 1.5 to less than 7.

Some chemistries have also included chelating agents to help remove cationic and anionic contamination from the wafer surface (International Application No. PCT/US98/02794) but chelating agents such as citric acid, gallic acid, and catechol among others, can be aggressive toward the aluminum oxide that covers the Al metal lines. Studies by Ohman and Sjoberg show that the strong complexing ability of citric ions can increase the aluminum oxide solubility and thereby expose the metal to further corrosion, by factors of 166 and 468 at pH 5 and 6 (see Ohman et al., J. Chem. Soc., Dalton Trans. (1983), p. 2513). Other resist-remover chemistries, such as those in U.S. Pat. No. 5,792,274, have included a salt of hydrogen fluoride combined with a water-soluble organic solvent and water at a pH of 5 to 8.

It is difficult to balance effective plasma etching residue removal and corrosion inhibition because chemical compositions of the plasma etching residues are generally similar to those of the metal layers or oxide layers on the substrate. The alkanolamine used in the prior art cleaning compositions was oftentimes found to attack both the plasma etching residues and the substrate metal layers in the presence of water. Water is often added as a contaminant, for example from the atmosphere, from wet components, and the like, and may even be released from certain photoresist structures during dissolution. The problem of water-cleaning composition induced corrosion has resulted in manufacturers resorting to alcohol or other solvent, for example isopropyl alcohol, to remove the cleaner.

Moreover, if a post-cleaner rinse such as isopropyl alcohol was not used, the corrosion could be very severe.

On the other hand, a composition for exfoliation solution used in removing resist residues is disclosed in Japanese Patent Laid Open No. H9-197681. The disclosed composition has an anti-corrosion effect as to the metal membranes on substrates and peripheral devices. The composition contains: a salt formed of hydrofluoric acid and a base without containing metal, a water soluble organic solvent, water, and at least one anti-corrosion agent such as aromatic hydroxy compounds, acetylene alcohol, organic compounds and anhydrides thereof containing carboxyl base, triazole compound, and sugars. The pH of the composition is ranging from 5 to 8. However, although the composition shows certain level of anti-corrosion effect, exfoliation power with regard to resist residues is reported to be very low. Thus, the composition may not be fully effective when used for the substrates subjected to extra fine patterning. In addition, its anti-corrosion effect may be insufficient to protect the substrate metal layers.

In addition to completely removing the resist material, particularly with the introduction of submicron process techniques to form wafers, there is a demand for cleaning technology for removing etching residue remaining following resist removal without corroding the substrates. Unfortunately, it has been found that no one cleaner is universal, in that it can clean the required materials without adversely affecting or hindering subsequent manufacturing operation or process steps involving the substrate. The requirement for a cleaning solution to remove photoresist and other residue of various types of metals, such as aluminum, aluminum/silicon/copper, titanium, titanium nitride, titanium/tungsten, tungsten, silicon oxide, polysilicon crystal, low-k materials, etc., presents a need for more effective cleaning chemistry in the processing area.

### SUMMARY OF THE INVENTION

One aspect of the invention relates to an exfoliation agent composition containing: (a) a salt formed of hydrofluoric acid and a base containing no metal ion; (b) one or more water soluble organic solvents; (c) a sugar alcohol; (d) water; and optionally (e) hydrofluoric acid. Advatnageously, the pH of the composition can be above about 8. In one embodiment, the composition can further contain a surfactant, preferably in an amount sufficient to improve the wetting property of the composition.

In one embodiment, the composition contains: 0.001-1% by weight of the composition of the salt formed of hydrofluoric acid and a base containing no metal ion; 50-98% by weight of the composition of the water soluble organic solvents; 0.01-10% by weight of the composition of the sugar alcohol; and the balance of water. In another embodiment, the composition contains: 0.005-0.5% by weight of the composition of the salt formed of hydrofluoric acid and a base containing no metal ion; 60-95% by weight of the composition of the water soluble organic solvents; 0.05-5% by weight of the composition of the sugar alcohol; and the balance of water. In yet another embodiment, the composition contains: 0.05-0.3% by weight of the composition of the salt formed of hydrofluoric acid and a base containing no metal ion; 75-95% by weight of the composition of the water soluble organic solvents; 0.1-3% by weight of the composition of the sugar alcohol; and the balance of water.

In an alternate embodiment, the composition contains: 0.001-1% by weight of the composition of the salt formed of hydrofluoric acid and a base containing no metal ion; 50-98% by weight of the composition of the water soluble organic solvents; 0.01-10% by weight of the composition of the sugar alcohol; 0.001-1% by weight of the composition of hydrofluoric acid; and the balance of water. In another alternate embodiment, the composition contains: 0.005-0.5% by weight of the composition of the salt formed of hydrofluoric acid and a base containing no metal ion; 60-95% by weight of the composition of the water soluble organic solvents; 0.05-5% by weight of the composition of the sugar alcohol; 0.005-0.5% by weight of the composition of hydrofluoric acid; and the balance of water. In still another alternate embodiment, the composition contains: 0.05-0.3% by weight of the composition of the salt formed of hydrofluoric acid and a base containing no metal ion; 75-95% by weight of the composition of the water soluble organic solvents; 0.1-3% by weight of the composition of the sugar alcohol; 0.05-0.3% by weight of the composition of hydrofluoric acid; and the balance of water.

In one embodiment, the salt formed of hydrofluoric acid and a base containing no metal ion can be ammonium fluoride. In another embodiment, the sugar alcohol can be xylitol. In yet another embodiment, the pH of the composition can be from about 8.5 to about 10.

Another aspect of the invention relates to a method of exfoliating the resist residues resulting from dry etching and plasma ashing, comprising: providing a substrate with resist residues resulting from dry etching and plasma ashing; contacting the substrate with a composition according to the invention for a time and at a temperature sufficient to cause the composition to substantially remove the resist residues; and rinsing the substrate.

### DETAILED DESCRIPTION OF THE INVENTION

Semiconductor elements such as IC, LSI, and those used in the liquid crystal display devices are manufactured according to the following steps. First, membranes of conductive metals such as aluminum and aluminum alloy and insulating membranes of SiO₂ are formed upon silicon or glass substrates. After having pasted host resist evenly upon membranes, resist patterns are formed by photo lithography process. Using the patterns thus formed as a mask, dry etching of the conductive metal and/or insulating membranes is conducted, and unnecessary resists must be removed therefrom to complete micro circuits.

The present invention encompasses a composition for exfoliation agent comprising: (a) a salt formed of hydrofluoric acid and a base containing no metal ion; (b) one or more water soluble organic solvents; (c) a sugar alcohol; and (d) water, wherein the pH of the said composition is above 8, and the composition is effective in removing resist residue.

The composition of the present invention contains a salt formed of hydrofluoric acid and a base containing no metal ion. Examples of “a base containing no metal ion” include, but are not limited to, organic amines such as primary, secondary, or tertiary aliphatic amine, cycloaliphatic amine, heterocyclic amine, ammonium, and alkyl quaternary ammonium base.

The term “primary aliphatic amine” includes, but is not limited to, methylamine, ethylamine, propylamine, butylamine, monoethanolamine (ethanolamine), monoisopropanolamine, and 2-(2-aminoethylamino)-ethanolamine.

The term “secondary aliphatic amine” includes, but is not limited to, dimethylamine, diethylamine, dipropylamine, dibutylamine, diethanolamine, N-methylethanolamine, and N-ethylethanolamine.

The term “tertiary aliphatic amine” includes, but is not limited to, trimethylamine, triethylamine, tripropylamine, tributylamine, triethanolamine, N,N-dimethylethanolamine, N,N-dimethylethanolamine, N-methyldiethanolamine, and N-ethyldiethanolamine.

The term “cycloaliphatic amine” includes, but is not limited to, cyclohexylamine, and dicyclohexylamine.

The term “heterocyclic amine” includes, but is not limited to, pyrrole, pyrrolidine, pridine, morpholine pyrazine, piperidine, oxazol, and thazole.

The term “alkyl quaternary ammonium base” includes, but is not limited to, tetramethylammonium hydroxide, and (2-hydroxyethyl) trimethylammonium hydroxide.

Preferably, compounds such as ammonium, monoethanolamine, and tetramethylammonium hydroxide, or mixtures thereof are appropriately used to provide the base containing no metal ion. A more preferred compound is ammonium.

As for the “salt formed of hydrofluoric acid and a base containing no metal ion”, ammonium fluoride or aqueous solution containing ammonium fluoride may be preferably used.

In the present invention, if the amount of the salt formed of hydrofluoric acid and a base containing no metal ion contained in the composition is less than 0.001% by weight of the composition, the resist residue exfoliation power of the composition is too weak, but if the amount is 1% by weight of the composition or more, the corrosive power of the composition against wiring materials and insulation membranes is too strong. Thus, a preferred composition would comprise the salt formed of hydrofluoric acid and a base containing no metal ion at a concentration from about 0.001% by weight of the composition to about 1% by weight of the composition. A more preferred composition would comprise the salt formed of hydrofluoric acid and a base containing no metal ion at a concentration from about 0.005% by weight of the composition to about 0.5% by weight of the composition. An even more preferred composition would comprise the salt formed of hydrofluoric acid and a base containing no metal ion at a concentration from about 0.05% by weight of the composition to about 0.3% by weight of the composition.

The composition of the present invention also contains one or more water soluble organic solvents as its component. Examples of “water soluble organic solvent” include, but are not limited to: amides such as N,N-dimethylformamide, N,N-dimethylacetaamide, N,N-diethylformamide, N,N-diethylacetamide, N-methylformamide, and N-methylacetamide; pyrrolidones such as N-methyl-2-pyrrolidone, N-ethyl-2-pyrrolidone, and N-hydroxyethyl-2-pyrrolidone; imidazolidinones such as 1,3-dimethy-2-imidazolidinone, and 1-3-diethyl-2-imidazolidinone; alkyl ureas such as tetramethyl urea, and tetraethyl urea; polyhydric alcohols and their derivatives such as ethylene glycol, ethylene glycol mono-methyl ether, ethylene glycol mono-ethyl ether, ethylene glycol mono-butyl ether, ethylene glycol mono-methyl ether acetate, ethylene glycol mono-ethyl ether acetate, diethylene glycol, diethylene glycol mono-methyl ether, diethylene glycol mono-ethyl ether, diethylene glycol mono-propyl ether, diethylene glycol mono-butyl ether, triethylene glycol mono-methyl ether, propylene glycol, propylene glycol mono-methyl ether, propylene glycol mono-ethyl ether, propylene glycol mono-butyl ether, dipropylene glycol mono-methyl ether, dipropylene glycol mono-ethyl ether, dipropylene glycol mono-propyl ether, dipropylene glycol mono-butyl ether, diethylene glycol dimethyl ether, diethylene glycol diethyl ether, diethylene glycol dipropyle ether, diethylene glycol diethyl ether, diethylene glycol dipropyl ether, diethyl glycol dibutyl ether, and triethylene glycol dimethyl ether; sulfoxides such as dimethyl sulfoxide, and diethyl sulfoxide; lactones such as y-butyrolactone, and α-valerolactone; oxycarbonic acid derivatives such as methyl lactate, ethyl lactate, propyl lactate, and butyl lactate; and oxazolidinones such as 3-methyl-2-oxazolidinone, and 3-ethyl-2-oxazolidinone.

The water soluble organic solvents mentioned above may be used alone or in combination of two or more. To obtain improved resist residue exfoliation effect, it is preferred to use a combination of two or more of the water soluble organic solvents.

In one preferred embodiment, the composition contains the water soluble organic solvents at a concentration from about 50% by weight of the composition to about 98% by weight of the composition. In a more preferred embodiment, the composition contains the water soluble organic solvents at a concentration from about 60% by weight of the composition to about 95% by weight of the composition. In an even more preferred embodiment, the composition contains the water soluble organic solvents at a concentration from about 75% by weight of the composition to about 95% by weight of the composition.

The composition of this invention also contains a sugar alcohol as its component. The sugar alcohol used in the present invention does not decrease exfoliation of resists residues while providing excellent anti-corrosion effect. Examples of the “sugar alcohol”, include, but are not limited to, traitol, erythritol, adonitol, xylitol, teritol, idetol, and dulcitol. A preferred sugar alcohol is xylitol.

In one preferred embodiment, the composition contains the sugar alcohol at a concentration from about 0.01% by weight of the composition to about 10% by weight of the composition. In a more preferred embodiment, the composition contains the sugar alcohol at a concentration from about 0.05% by weight of the composition to about 5% by weight of the composition. In an even more preferred embodiment, the composition contains the sugar alcohol at a concentration from about 0.1% by weight of the composition to about 3% by weight of the composition.

The pH of the composition of the present invention is above 8. A pH lower than 8 is reported to be undesirable because the exfoliation effect against resist residues is reduced at such a pH. A preferred composition would have a pH ranging from about 8.5 to about 10.

The pH can be adjusted by using pH adjusting agents including, but not limited to: inorganic acids such as sulfuric acid, nitric acid, hydrochloric acid, and phosphoric acid; organic acids such as acetic acid, propione acid, oxalic acid, and citric acid; ammonium; organic amine; and low-grade quaternary ammonium base. The amount of said pH adjusting agent added can be readily determined by the persons of ordinary skill in the art according to the specific components of the composition.

To improve the wetting property of the composition of the present invention, and to further improve exfoliation property, a surfactant may be added without any adverse effect. Any surfactant known in the art, including cation system, nonion system, anion system, or fluorine system can be used.

There is no specific limit as to the temperature at which the composition of the present invention may be used. Both the exfoliation time and exfoliation temperature can be readily determined by the persons of ordinary skill in the art based upon factors such as the condition of resist residues and specific components to be used in the composition.

One aspect of this invention is directed to a method of exfoliating the resist residues resulting from dry etching and plasma ashing, comprising: providing a substrate with a resist residues resulting from dry etching and plasma ashing; contacting the substrate with the composition of this invention for a time and at a temperature sufficient to cause the composition to substantially remove the resist residues; and rinsing the substrate.

Examples of the “substrate” include, but are not limited to: silicone; poly-silicone; silicone oxide membrane; silicone nitride membrane; aluminum alloys such as aluminum, aluminum/copper, and aluminum/silicone/copper; titanium alloys such as titanium, titanium nitride, and titanium/tungsten; semiconductor materials such as tungsten, tantalum, and copper; compound semiconductor materials such as gallium/arsenic, gallium/phosphorus, and indium/phosphorus; and LCD materials such as a-silicone and low temperature poly-silicone.

The rinsing step can be carried out by using water soluble organic solvents, mixture of water soluble organic solvent and extra pure water, or extra pure water only.

Another part of this invention is directed to a composition for exfoliation agent comprising: (a) a salt formed of hydrofluoric acid and a base without metal ion; (b) one or more water soluble organic solvents; (c) a sugar alcohol; (d) water; and (e) hydrofluoric acid, wherein the pH of the composition is above 8, and the composition is effective in removing resist residue. This aspect of the present invention encompasses all components described herein above, but additionally contains hydrofluoric acid as its component. Accordingly, the disclosure with regard to the identical components described herein above, i.e., a salt formed of hydrofluoric acid and a base without metal ion, one or more water soluble organic solvents, a sugar alcohol and water, remains valid for this aspect of the present invention as well.

This aspect of the invention, however, additionally contains hydrofluoric acid as its component. In the present invention, if the amount of hydrofluoric acid contained in the composition is less than 0.001% by weight of the composition, the exfoliation power of the composition is too weak, and corrosive power against wiring materials is too strong if the amount of the same is more than 1% by weight of the composition. Thus, a preferred composition would contain the hydrofluoric acid at a concentration from about 0.001% by weight of the composition to about 1% by weight of the composition. A more preferred composition would contain the hydrofluoric acid at a concentration from about 0.005% by weight of the composition to about 0.5% by weight of the composition. An even more preferred composition would contain the hydrofluoric acid at a concentration from about 0.05% by weight of the composition to about 0.3% by weight of the composition.

### EXAMPLES

Certain embodiments of this invention, as well as certain advantages of this invention, are illustrated by the following non-limiting examples. Although limited number of examples are disclosed herein, it will be apparent to those skilled in the art that many modifications, both to materials and methods, may be practiced without departing from the purpose and interest of this invention.

Preparation of Sample Substrate

A sample substrate of Al alloy circuit element was prepared in the following steps. A silicon oxide membrane upon a silicon substrate was formed by heat oxidation. Then, a membrane of barrier metal comprising titanium nitride (TiN), Al/Cu wiring, and another barrier metal membrane over the said titanium nitride were formed by magnetron sputtering. Next, resist was coated by spin coater, then a resist pattern was formed by exposure and development processes. Dry etching was conducted using this resist pattern as a mask and BCl₃/Cl 2 gas. After dry etching is completed, remaining resist pattern was treated by plasma ashing at 250° C., using a parallel flat sheet type RIE device. Upon completion of the plasma ashing, resist residues remained upon side walls of the pattern and upon the upper TiN membrane.

Exfoliation of Resist Residues and Anti-Corrosion Effect

The compositions to be used for resist residue exfoliation were prepared as shown in Tables 1-1 and 1-2 below. Execution Examples 1-6 are compositions prepared according to the disclosure of the present invention. In Comparative Examples 1-5, samples were prepared with one or more components and/or conditions not in accord with the disclosure of the present invention. DGME denotes diethylenglycol monomethylether; DGBE denotes diethyleneglycol monobutylether; DPGME denotes dipropyleneglycol monomethylether; DMAC denotes dimethylacetamide; DMF denotes dimethylformamide; and NMP denotes N-methyl-2-pyrolidone. The unit is % by weight of the composition.

TABLE 1-1

| | Execution | Execution | Execution | Execution | Execution | Execution |
|---|---|---|---|---|---|---|
| | Example 1 | Example 2 | Example 3 | Example 4 | Example 5 | Example 6 |
| Ammonium | 0.15 | 0.05 | 0.3 | 0.3 | 0.1 | — |
| fluoride | | | | | | |
| Acid ammonium | — | — | — | — | — | 0.5 |
| fluoride | | | | | | |
| DGME | 32.0 | — | — | — | — | — |
| DGBE | — | 51.0 | 51.0 | — | 32.0 | 49.0 |
| DPGME | — | — | — | 51.0 | — | — |
| DMAC | 55.0 | 40.0 | — | — | 55.0 | 38.0 |
| DMF | — | — | — | 40.0 | — | — |
| NMP | — | — | 40.0 | — | — | — |
| Xylitol | 0.5 | 0.5 | 1.0 | 1.0 | 0.3 | 0.8 |
| Water | balance | balance | balance | balance | balance | balance |
| pH | 9.0 | 9.0 | 8.6 | 8.7 | 8.4 | 8.8 |

TABLE 1-2

| | Comparative | Comparative | Comparative | Comparative | Comparative |
|---|---|---|---|---|---|
| | Example 1 | Example 2 | Example 3 | Example 4 | Example 5 |
| Ammonium fluoride | 0.15 | 0.15 | 0.15 | 0.15 | — |
| Acid ammonium | — | — | — | — | 0.5 |
| fluoride | | | | | |
| DGME | 32.0 | — | 32.0 | 32.0 | 32.0 |
| DGBE | — | 87.5 | — | — | — |
| DPGME | — | — | — | — | — |
| DMAC | 55.0 | — | 55.0 | 55.0 | 55.0 |
| DMF | — | — | — | — | — |
| NMP | — | — | — | — | — |
| Sugar, Sugar alcohol | — | — | D-sorbitol, | Sucrose, | — |
| | | | 0.5 | 0.5 | |
| Water | balance | balance | balance | balance | balance |
| pH | 8.9 | 7.8 | 7.4 | 7.1 | 8.6 |

The sample substrates were incubated in the compositions prepared as above at 23° C. for 5 to 10 minutes. The substrates were then rinsed by extra pure water, followed by nitrogen gas dry. The amount of resist residues remained upon the sample substrates and the degree of corrosion upon the surface of Al alloy wiring were then evaluated. Evaluations were conducted by surveying the surfaces of sample substrates after treatments using a scanning electron microscope (SEM). The results are shown in Table 2-1 and 2-2.

TABLE 2-1

| | Execution | Execution | Execution | Execution | Execution | Execution |
|---|---|---|---|---|---|---|
| | Example 1 | Example 2 | Example 3 | Example 4 | Example 5 | Example 6 |
| Immersion time | 5 | 5 | 10 | 5 | 5 | 5 |
| (min) | | | | | | |
| Condition of resist | ◯ | ◯ | ◯ | ◯ | ◯ | ◯ |
| residues remained | | | | | | |
| Condition of | ◯ | ◯ | ◯ | ◯ | ◯ | ◯ |
| Al corrosion | | | | | | |

TABLE 2-2

| | Com- | Com- | Com- | Com- | Com- |
|---|---|---|---|---|---|
| | parative | parative | parative | parative | parative |
| | Example 1 | Example 2 | Example 3 | Example 4 | Example 5 |
| Immersion | 5 | 5 | 5 | 5 | 5 |
| time | | | | | |
| Condition | ◯ | Δ | X | X | ◯ |
| of resist | | | | | |
| residues | | | | | |
| remained | | | | | |
| Condition of | Δ | ◯ | ◯ | ◯ | X |
| Al corrosion | | | | | |

The symbols used in Table 2-1 and 2-2 represent:
Conditions of Resist Residue Exfoliation:
◯ . . . Resist residues were completely exfoliated.
Δ . . . Resist residues partially remained.
X . . . Majority of resist residues remained.
Anti Corrosion Effect Against Al Alloy:
◯ . . . No corrosion was observed.
Δ . . . Partial corrosion was observed.
X . . . Considerable corrosion was observed.

Table 2-1 and 2-2 show that the compositions in Comparative Examples 1-5 possess either a good exfoliation effect or a good anti-corrosion effect. None of the compositions in Comparative Examples 1-5 showed, however, both good exfoliation and anti-corrosion effect. On the other hand, the compositions in Execution Examples 1-6 showed both excellent resist residue exfoliation effect and anti-corrosion effect.

1. A composition for exfoliation agent comprising:
(a) a salt formed of hydrofluoric acid and a base containing no metal ion;
(b) one or more water soluble organic solvents;
(c) a sugar alcohol; and
(d) water,
wherein the pH of the composition is above 8, and the composition is effective in exfoliating resist residues.

2. The composition of claim 1, wherein the said composition comprises:
0.001-1% by weight of the composition of the salt formed of hydrofluoric acid and a base containing no metal ion;
50-98% by weight of the composition of the water soluble organic solvents;
0.01-10% by weight of the composition of the sugar alcohol; and
balance of water.

3. The composition of claim 1, wherein the said composition comprises:
0.005-0.5% by weight of the composition of the salt formed of hydrofluoric acid and a base containing no metal ion;
60-95% by weight of the composition of the water soluble organic solvents;
0.05-5% by weight of the composition of the sugar alcohol; and
balance of water.

4. The composition of claim 1, wherein the said composition comprises:
0.05-0.3% by weight of the composition of the salt formed of hydrofluoric acid and a base containing no metal ion;
75-95% by weight of the composition of the water soluble organic solvents;
0.1-3% by weight of the composition of the sugar alcohol; and
balance of water.

5. The composition of claim 1, wherein the salt formed of hydrofluoric acid and a base containing no metal ion is ammonium fluoride.

6. The composition of claim 1, wherein the sugar alcohol is xylitol.

7. The composition of claim 1, wherein the pH of the said composition is from about 8.5 to about 10.

8. The composition of claim 1, wherein the composition further comprises a surfactant in an amount sufficient to improve the wetting property of the composition.

9. A method of exfoliating the resist residues resulting from dry etching and plasma ashing, comprising:
providing a substrate with resist residues resulting from dry etching and plasma ashing;
contacting the substrate with the composition of claim 1 for a time and at a temperature sufficient to cause the composition to substantially remove the resist residues; and
rinsing the substrate.

10. A composition for exfoliation agent comprising:
(a) a salt formed of hydrofluoric acid and a base without metal ion;
(b) one or more water soluble organic solvents;
(c) a sugar alcohol;
(d) water; and
(e) hydrofluoric acid,
wherein the pH of the composition is above 8, and the composition is effective in exfoliating resist residues.

11. The composition of claim 10, wherein the said composition comprises:
0.001-1% by weight of the composition of the salt formed of hydrofluoric acid and a base containing no metal ion;
50-98% by weight of the composition of the water soluble organic solvents;
0.01-10% by weight of the composition of the sugar alcohol;
0.001-1% by weight of the composition of hydrofluoric acid; and
balance of water.

12. The composition of claim 10, wherein the said composition comprises:
0.005-0.5% by weight of the composition of the salt formed of hydrofluoric acid and a base containing no metal ion;
60-95% by weight of the composition of the water soluble organic solvents;
0.05-5% by weight of the composition of the sugar alcohol;
0.005-0.5% by weight of the composition of hydrofluoric acid; and
balance of water.

13. The composition of claim 10, wherein the said composition comprises:
0.05-0.3% by weight of the composition of the salt formed of hydrofluoric acid and a base containing no metal ion;
75-95% by weight of the composition of the water soluble organic solvents;
0.1-3% by weight of the composition of the sugar alcohol;
0.05-0.3% by weight of the composition of hydrofluoric acid; and
balance of water.

14. The composition of claim 10, wherein the salt formed of hydrofluoric acid and a base containing no metal ion is ammonium fluoride.

15. The composition of claim 10, wherein the sugar alcohol is xylitol.

16. The composition of claim 10, wherein the pH of the said composition is from about 8.5 to about 10.

17. The composition of claim 10, wherein the composition further comprises a surfactant in an amount sufficient to improve the wetting property of the composition.

18. A method of exfoliating the resist residues resulting from dry etching and plasma ashing, comprising:
providing a substrate with resist residues resulting from dry etching and plasma ashing;
contacting the substrate with the composition of claim 10 for a time and at a temperature sufficient to cause the composition to substantially remove the resist residues; and
rinsing the substrate.
